(19) United States
(12) Patent Application Publication
(10) Pub. No.: US 2017/0304525 A1
(43) Pub. Date: Oct. 26, 2017

(54) SYSTEM AND METHOD FOR PROVIDING AN INJECTION

(71) Applicant: VETTER PHARMA-FERTIGUNG GmbH & Co. KG, Ravensburg (DE)

(72) Inventor: Tilman ROEDLE, Wolfegg (DE)

(73) Assignee: VETTER PHARMA-FERTIGUNG GmbH & Co. KG, Ravensburg (DE)

(21) Appl. No.: 15/517,435

(22) PCT Filed: Oct. 6, 2015
(86) PCT No.: PCT/EP2015/072998

§ 371 Date: Apr. 6, 2017

(30) Foreign Application Priority Data
Oct. 8, 2014 (DE) 10 2014 220 365.1

(51) Int. Cl. A61M 3/00; A61J 1/20; A61J 1/06

(52) U.S. Cl. CPC A61M 3/005; A61J 1/2096; A61J 1/2093; A61J 1/201; A61J 1/067; A61J 1/2048; A61J 1/20

(57) ABSTRACT

A system for preparing an injection includes a first container with a first cavity and a rigid outer wall and two ends, a movable stopper being provided on one side of the first cavity, which sealingly closes this cavity and is displaceable inside the first container and comprising a second container, which encloses a second cavity, one container comprising a first substance, and the other container comprising a second substance. The second container comprises an at least regionally elastic wall and in this way has a variable volume. The second container is designed such that a pressure can be built inside the second container by way of the at least regionally elastic wall. The second container is a tube.

The invention relates to a system for preparing an injection according to the preamble of claim 1, moreover to a combination of a syringe or carpule with a tube according to claim 12, and to a method for preparing an injection according to the preamble of claim 13.

Systems and methods of the type addressed here are known. They comprise a first container, which is designed as a cylindrical body having a first cavity and represents a syringe or carpule, for example. A powdered substance, and in particular a lyophilizate, representing a medical substance is present therein. The purpose of this substance is to be injected into a patient. To this end, it is necessary to dissolve the powdered substance. It is known to provide the first container with an injection cannula so as to take in a liquid from a second container having a second cavity and thereby dissolve the powdered substance present in the first cavity of the cylindrical body of the first container. The generated solution can then be administered to a patient. In conventional systems, the second container is made of glass or a solid transparent plastic material. In general, like the first container, this container is not filled completely and includes a volume of air. In this way, it is possible to transfer the solvent, which is also referred to as a diluent, from the cavity of one container into the cavity of the other container so as to dissolve the powdered substance. It has been shown that the dissolution process is time-consuming when the powdered substance is difficult to dissolve. It is also possible that the powdered substance does not dissolve completely, which can lead to problems with the application or injection. Systems that comprise a first and a second container are also known, however in contrast to what is described above, both containers comprise a liquid. To prepare an injection, these two liquids must be mixed with one another. It is essential that these liquids are mixed completely, so that the components of the two liquids are essentially distributed homogeneously before the mixture is applied. It is found with some medical liquids that the complete and homogeneous mixing thereof can only be achieved with difficulty. This is the case in particular with vaccines and vitamin products.

It is thus the object of the invention to create a system that avoids this disadvantage.

To achieve this object, a system for preparing an injection is proposed, which comprises the features of claim 1. The system comprises a first container, which encloses a first cavity and comprises a rigid outer wall and two ends, wherein a movable stopper is provided on one side of the first cavity, which sealingly closes this cavity and is displaceable inside the container. The system comprises a second container enclosing a second cavity. The system is characterized by a coupling device, which comprises a first coupling element provided on the first container and a second coupling element provided on the second container, and is characterized in that the two containers can be connected to one another by way of the coupling elements such that the cavities thereof are in fluid connection with one another. The system is furthermore characterized in that the second container has a variable volume. As a result of the fluid connection between the two cavities in the first and second containers, it is possible to thoroughly mix the first substance with the liquid substance. The variable volume of the second container helps to ensure that the thorough mixing can take place very intensively.

The system proposed here is characterized in that the second container comprises a wall that is elastic at least in regions, thus having a variable volume. In this way, the system is configured such that the second container is expanded by the introduction of a gas volume and/or liquid volume, whereby the wall addressed here is elastically extended at least in regions. Due to the inherent elasticity of the wall, the interior of the second container is under such an internal pressure that a substance provided here can be discharged under pressure, and thereby reaches the first container.

For thoroughly mixing the two substances, the liquid substance present in the second container is transferred into the first container. The substance can flow completely into the first container since the elastic outer wall of the second container can be curved inwardly, whereby the volume of the cavity encased in the second container can be reduced. If the volume in the second container thus increases by the expansion of the regionally elastic outer wall when a liquid is introduced, the same, as mentioned, can also be decreased. This results in considerably better thorough mixing of the two substances than is the case with conventional systems.

In this way, good thorough mixing of the second substance, present in the second container and expelled therefrom, with the first substance in the first container takes place. This applies in the case that a liquid or powdered substance is present in the first container, on which the second substance from the second container impinges as a result of the positive pressure that is built by the at least one elastic wall region. So even if a powder is present in the second container, this is transferred into the first container by the positive pressure and is blown into a liquid.

The system described here, in which the second container is designed as a tube, is characterized in that a cost-effective implementation is provided. Here, a tube refers to an element having a hollow, and in particular tubular, base body, which is closed at one end. If the tube is implemented from a plastic material, the basic body can be welded closed, either by way of heat, frictional heat or by way of ultrasonic weld bonding. It is also possible without difficulty to use other materials and sealing methods.

The opposite other end of the base body is preferably designed as an integral cover of the same and includes a closable opening, which is preferably surrounded by a preferably cylindrical shoulder. The cover preferably has a slightly more stable design than the side walls of the base body. The cylindrical shoulder can preferably be provided with an external thread, to which a screw cap comprising an internal thread is applied. Plug or compression closures are also conceivable.

The crucial factor is that the production of tubes using methods known today can be achieved in a simple and extremely cost-effective manner, even if the outer wall of the tube has an elastic design at least in regions and preferably is opaque or transparent.

When configuring the cylindrical shoulder in the region of the cover with an external thread, it is very easily possible to couple the tube to the first container, for example by way of a threaded joint, and to implement a fluid connection between the interiors of the first container and the tube. The external thread of the tube can also be adapted to an internal thread of a Luer connection, whereby the tube can be readily used with conventional syringes or the like.

A preferred exemplary embodiment of the system is characterized in that the first substance is a lyophilizate, and the second substance is a solvent for the lyophilizate, referred to as a diluent. By accommodating the lyophilizate and the solvent in separate cavities in this system, an excellent storage life of the lyophilizate is achieved.

A further exemplary embodiment of the system is characterized in that lyophilizate is present in the first container, and a solvent for the lyophilizate is present in the second container.

In a particularly preferred exemplary embodiment of the system, it is provided that this is configured and designed such that an amount of a second substance is provided in the second container for an amount of a first substance in the first container, so as to provide an amount of an injection solution for an injection. It is thus possible to match the containers comprising the substances to be provided for an injection to one another, whereby the system is very easy to handle.

In particular, the volume of the first container is matched to the amount of the first substance and/or the volume of the second container is matched to the amount of the second substance. This results in the advantage that the system has a very compact design.

An exemplary embodiment of the system in which a number of containers comprising differing amounts of a substance is provided is particularly preferred. These containers can be combined with other containers that comprise differing amounts of another substance, wherein a first container comprising a first substance is brought in fluid connection with a second container comprising a second substance so as to mix the two substances with one another. It is a characteristic of this system that the amount of the second substance is matched exactly to the amount of the first substance so as to provide an injection solution for an injection. For example, liquids are provided in the two containers, wherein the amount in the first container is matched to the amount in the second container such that exactly a certain mixing ratio of the two liquids is achieved. This is relevant in order to provide an injection solution having a certain concentration for an injection. In addition, this aspect is relevant for mixing two liquids with one another, for example, in which the quantity ratios must be matched to one another to achieve complete mixing.

It is also possible to provide a certain amount of a first substance in a first container, and to combine this with different containers so as to achieve an injection solution having differing concentrations. For example, a lyophilizate may be present in the first container. In the second container, the amount of liquid provided is at least the amount required for dissolving or activating the lyophilizate. When the first container is combined with a second container having a larger solvent amount, it is moreover possible to generate an injection solution having a different concentration from an amount of a lyophilizate.

The system designed in such a way is also characterized in that lyophilizates of differing types, which is to say first substances, are provided in a number of first containers, the lyophilizates differing in that these require differing amounts of diluents, which is to say solvents, for complete dissolution. It is easily possible with the system described here to combine second containers comprising differing amounts of diluents or solvents with the first containers comprising differing lyophilizates. A second container comprising a certain amount of a diluent can be provided for a first container comprising a first lyophilizate so as to provide an injection solution. A different amount of solvent can be supplied to a container comprising a second lyophilizate by combining a different second container comprising a larger or smaller amount of solvent.

The system defined here can thus be easily adapted to different application cases, be it to the use of different substances for administration to a patient or for the use of injection solutions having differing concentrations of the ingredients. Essentially a modular system composed of two containers is thus provided, wherein the two containers comprise differing substances and/or have differing volumes for receiving substances, so as to provide an injection solution for an injection in a simple and optimal manner.

The system described here is thus characterized by a flexible usage option.

A particularly preferred exemplary embodiment of the system is characterized in that the first container is a syringe or carpule, and the second container is a tube. This comprises an outer wall that is elastic at least in regions, so that the same can expand when the two substances in the first and second cavities are mixed and be contracted, whereby the inside volume thereof is reduced. The system defined here is characterized in that a conventional single-chamber syringe or a single-chamber carpule comprising a lyophilizate can be easily coupled to the second container so as to dissolve the lyophilizate.

Further exemplary embodiments are apparent from the related dependent claims.

It is furthermore an object of the invention to create a combination of a syringe or carpule and a tube which overcomes this disadvantage.

To achieve this object, a combination of a syringe or carpule and a tube comprising at least one regionally elastic wall is created. This is characterized in that the volume provided in the syringe or carpule and the volume provided in the tube are matched to one another such that the syringe is able to receive a defined amount of a first substance and the tube is able to receive a defined amount of a second substance. Due to the at least regionally elastic wall, it is possible to press at least a partial volume from the syringe or carpule into the tube, and thereby expand the wall against the restoring forces thereof provided by the elasticity. This causes the at least regionally elastic wall to contract again after a partial volume has been introduced from the first container, and the second substance in the tube to be introduced under pressure into the first container. A liquid thus impinges at a high speed and with high energy on a powder or a liquid in the first container, whereby optimal mixing of the substances takes place for generating an injection solution. Optimal refers to a state here in which a lyophilizate is dissolved completely by a solvent, without leaving residue, or that two liquids are mixed with one another such that the concentration of the two liquids in the entire volume of the resultant injection solution is the same. It shall be pointed out again here that it is also possible to spray a powder from the tube into a liquid, which is present in the syringe or carpule.

It is furthermore an object of the invention to create a method that avoids this disadvantage.

To achieve the object, a method for preparing an injection by way of a system of the type described here is proposed. The method comprises the features of claim 13 and the following steps: Initially, the two containers are connected to one another via the coupling device such that a fluid connection is implemented. Then, the liquid substance is transferred into the solid, preferably powdered, substance. This transfer process is very effective since the liquid substance is present in the container that has an at least regionally elastic outer wall. The elastic outer wall region can be expanded or retracted for transferring the liquid substance into the cavity comprising the solid substance, whereby as much of the liquid substance as possible reaches the solid substance. In a further step, the substances in the containers are mixed by way of shaking and/or moving a stopper provided in the one container. During the movement of the stopper, it is particularly advantageous that the second container comprises an outer wall that is elastic at least in regions. Due to the elastic outer wall, it is ensured that no significant counter-pressure builds during the movement of the stopper, which could impede the movement of the stopper.

One embodiment of the method, which is characterized in that a Luer connection is used as the first coupling element, which receives an external thread of a second coupling element, for coupling the two containers in a fluid-tight manner, is particularly preferred.

Further preferred embodiments of the method will be apparent from the related dependent claims.

The invention will be described in greater detail hereafter based on the drawings. In the drawings:

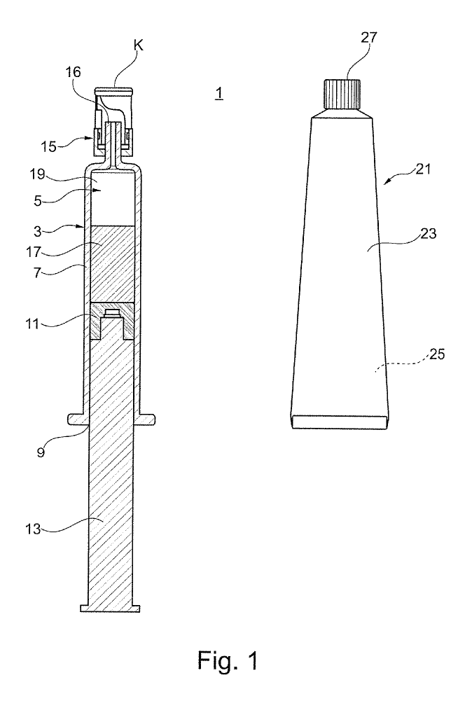

FIG. 1 shows a system for preparing an injection comprising a first and a second container;

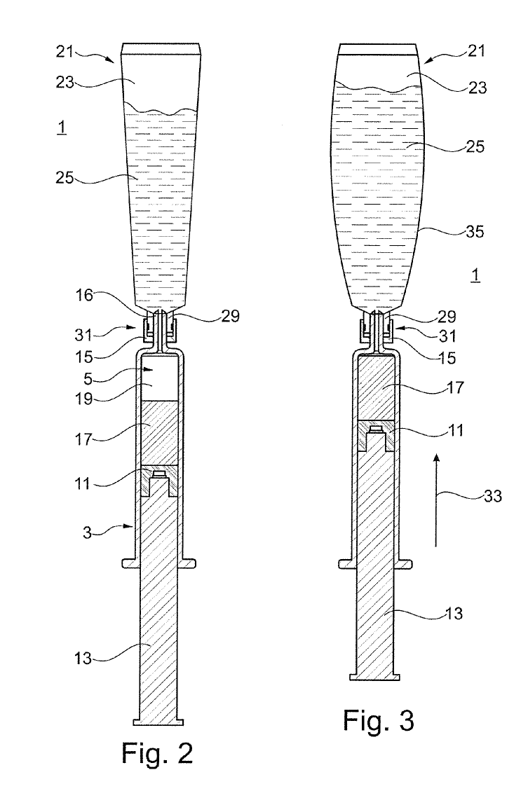

FIG. 2 shows the system according to claim 1 with coupled containers in a first functional position; and

FIGS. 3 to 5 show the system in further functional positions.

FIG. 1 shows a system 1 for preparing an injection comprising a first container 3, which encloses a first cavity 5 and comprises a rigid outer wall 7. The first container 3 has two ends, wherein at a first end an opening 9 is provided, via which a stopper 11 that is movable in the first cavity 5 can be introduced. By way of a plunger rod 13, which is coupled to the stopper 11, the stopper 11 can be displaced inside the first container along the longitudinal axis thereof, which is perpendicular here. The stopper 11 is designed so as to be sealingly seated against the inside surface of the outer wall 7, whereby the cavity 5 is sealingly closed with respect to the opening 9. A first coupling element 15 is attached at the end of the first container 3 located opposite the opening 9. At this end, the first container 3 comprises a shoulder 16 onto which the coupling element 15 is placed. The first container 3 is a syringe. In another exemplary embodiment of the container, a protrusion comprising a cap, which sealingly closes the cavity 5 of the container, may be formed at the end present here. The container can thus also be designed as a carpule, wherein a piercable stopper may be present at the end addressed here, but in particular also the option exists of connecting a further container via a coupling element so as to establish a fluid connection between the further container and the cavity 5 in the container. This will be addressed in greater detail hereafter.

The first container 3 comprises a first substance, which is accommodated in the cavity 5. For example, the first substance is a powder, and in particular a lyophilizate 17, which is to say a freeze-dried substance, which is present in powdered form. An air volume 19 is present above the lyophilizate 17. The first substance, however, can also be a liquid.

The system 1 represented here comprises a second container 21, which encloses a cavity 23. A second substance, for example a powder, is present therein, provided the first substance is a liquid, a liquid serving as a diluent when the first substance is a powder, and in particular a solvent 25 when the first substance is a lyophilizate. In principle, it shall be noted that differing substances are provided in the two containers 3 and 21, preferably a lyophilizate on the one hand and a solvent, referred to as a diluent, on the other hand. In the exemplary embodiment of the system 1 shown here, it is assumed that the lyophilizate 17 is present in the first container 3 and the solvent 25 is present in the second container 21. However, it is also possible to accommodate the lyophilizate 17 in the second container 21 and the solvent in the first container 3.

At one end, which is the upper end here, the second container 21 comprises a closure 27, which sealingly closes the cavity 23 of the second container 21.

A cap K, which is held by the coupling element 15 and is used to sealingly close the cavity 5 of the first container 3, is provided at the end of the container 3 located at the top in FIG. 1. In this way it is ensured that the substance accommodated in the cavity 5, which is the lyophilizate 17 here, is safely encased, and a long shelf life without contamination is ensured. The cap K, as is known, is preferably designed as a tamper-evident cap, so that manipulations on the cap are readily apparent to a user of the first container 3.

In FIG. 1, the two containers 3 and 21 of the system 1 are thus separate from one another, and each is sealingly closed, whereby the substances introduced into the associated cavities 5 and 23 of the containers 3 and 21 are safely encased.

When a substance encased in the containers 3 and 21, which is the lyophilizate 17 in the first container 3 here, is to be administered to a patient, the lyophilizate 17 must be dissolved to prepare an injection. This is carried out by way of the solvent 25 accommodated in the second container 21.

To be able to dissolve the lyophilizate 17, the two containers 3 and 21 must be brought in fluid connection with one another. So as to make this possible, the cap K must be removed from the first container 3, and the closure 27 must be removed from the second container 21. By removing the cap K, the first coupling element 15 on the first container 3 becomes accessible. Preferably, this is a Luer connection here, which is placed on the shoulder 16 or integrally formed onto the container 3 and comprises an internal thread.

When the closure 27 is removed from the second container 21, a second coupling element, which can cooperate with the first coupling element 15 on the first container 3, becomes accessible at the end of the second container 21 associated with the closure 27. In the event that the first coupling element 15 is designed as a Luer connection, the second coupling element 29 on the second container 21 is designed as a cylindrical shoulder comprising an external thread, which cooperates with the internal thread in the Luer shoulder. In this way, it is possible to screw the second container 21 onto the first container 3, wherein the first coupling element 15 sealingly receives the second coupling element. The two coupling elements thus form a coupling device 31, which ensures a tight fluid connection between the cavity 5 in the first container 3 and the cavity 23 of the second container 21.

The end of the second container 21 can also be closed by way of a membrane, which is covered by the closure 27. This has the advantage that the content of the second container is not yet freely accessible immediately after the closure 27, which can be designed as a screw cap, for example, has been removed. As a result of the membrane, protection against contamination of the content or against undesirable leaking of the same is ensured. After the closure 27 has been removed, the membrane can be pierced and torn open, for example by the shoulder 16 at the end of the first container 3. In this way, a fluid connection is then implemented between the two containers 3 and 21.

FIG. 2 shows the system 1 comprising the containers 3 and 21, which are coupled to one another by way of the coupling device 31 comprising the two coupling elements 15 and 29, in a first functional position. Identical parts are denoted by identical reference numerals, wherein in this regard reference is made to the description of FIG. 1 to avoid repetitions.

The first functional position of the system 1 shown in FIG. 2 is characterized in that a fluid connection between the cavity 5 in the first container 3 and the cavity 23 in the second container 21, which is shown in a longitudinal section here, is established via the coupling device 31, wherein the stopper 11 is in the starting position, which is also shown in FIG. 1, in the first container 3. The stopper 11 inside the first container is still in the starting position thereof.

FIG. 3 shows the system 1 comprising the two containers 3 and 21 in a second functional position. Identical parts are denoted by identical reference numerals, wherein in this regard reference is made to the description above.

In the second functional position according to FIG. 3, the stopper 11 has been displaced out of the position shown in FIGS. 1 and 2 and upward, according to the arrow 33, in the direction of the coupling device 31. Since the two containers 3 and 21 are in fluid connection, the air volume 19 in the first cavity 5, which is apparent in FIGS. 1 and 2, is displaced during an upward movement of the stopper 11, and reaches the second cavity 23 of the second container 21. It is possible for air to be present in the second cavity 23 even before the second container 21 is used, as is shown in FIG. 2; however, this is not absolutely necessary.

The second container 21 has a variable volume. It has been shown that the wall 35 of the second container 21 can expand at least in regions as soon as a positive pressure is built in the second cavity 23 by the introduced air volume 19. FIG. 3 shows the second container 21 in the expanded state. As is also apparent from FIGS. 1 and 2, the second container 21 is preferably designed as a tube here, which is made of an elastic, and in particular opaque or transparent, material, whereby the substance present in the cavity 23, which is the solvent 25 here, is visible. The material of the second container 21 is preferably designed such that the cavity 23 of the container is protected against UV light. The wall 25 is made of a material that does not change during storage of the system 1 and that, in particular, does not change the substance 25 present in the cavity 23 during storage. Preferably, it is provided that the wall 35 is coated on the inside surface thereof facing the cavity 23. It is also possible to implement the wall with two or multiple layers, so as to reduce the permeability thereof, among other things. An embodiment of the wall in which the interior layer facing the cavity 23 is resistant to the substance present in the cavity 23 is particularly preferred. The remaining material or the remaining layers of the wall 35 can then optionally be made of less expensive materials. Multi-layer walls can be implemented by way of coating, and also by way of multi-layer extrusion.

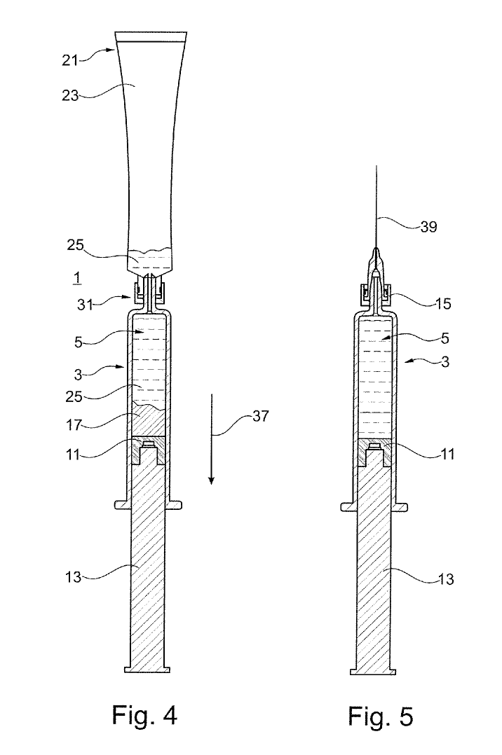

In the functional position shown in FIG. 3, the air volume 19 present in the cavity 5 was thus displaced by an upward displacement of the stopper 11 in the direction of the coupling device 31 into the cavity 23 of the second container 21, whereby this expands and has a larger inside volume than is the case in the first functional position according to FIG. 2. FIG. 4 shows a third functional position of the system 1. Identical parts are denoted by identical reference numerals, wherein in this regard reference is made to the description above.

It is apparent from the illustration according to FIG. 4 that the stopper 11 has been moved out of the position thereof shown in FIG. 3 and downward in the direction of the arrow 37 by way of the plunger rod 13, whereby the solvent 25 present in the cavity 23 of the second container 21 preferably completely reaches the cavity 5 of the first container 3, where it can mix with the lyophilizate 17 present there. A defined liquid volume is thus supplied to the lyophilizate. In this way, it is also possible to supply a liquid present in the first container 3 to a second liquid present in the second container 21.

By introducing the air volume 19, the second container 21 was expanded, as stated above, wherein the wall 35 thereof was extended. A positive pressure was thus created in the second container 21. If the stopper 11 is now pulled downward in the direction of the arrow 37, a negative pressure is created in the cavity 5 of the first container 3, which suctions the solvent 25 from the second container 21. This solvent impinges on the lyophilizate 17 at a high speed since, additionally, it is injected into the first container 3 by the positive pressure inside the second container 21. This results in excellent thorough mixing of the diluent with the powdered lyophilizate 17, or of the two liquids that may be present in the containers 3 and 21.

The stopper 11 is preferably pulled out of the position shown in FIG. 4 and further downward, however so as not to be pulled out of the first container 3. As a result, the substances present in the cavity 5 remain tightly encased, which is to say safe from contamination.

As a result of the further displacement of the stopper 11 downward in the direction of the arrow 37, which is to say beyond the position shown in FIG. 4, air is suctioned in from the second container 21, whereby the lyophilizate 17 can be easily dissolved in the first cavity 5 by shaking the two containers 3 and 21. It is also possible to displace the stopper upward very easily since the second container 21 has a variable volume, and thus does not offer strong resistance when the stopper 11 is being displaced in the direction of the coupling element 31.

This ensures that optimal thorough mixing of the substances, and in particular of the lyophilizate 17 and of the solvent 25, can take place in the first cavity 5 of the first container 3. Good thorough mixing is incidentally also provided when liquids are present in the two containers 3 and 21 that generally are relatively difficult to dissolve. This is often the case with vaccines and vitamin products, for example.

FIG. 5 finally shows the first container 3 of the system 1 in a further functional position. Identical parts are denoted by identical reference numerals, wherein in this regard reference is made to the description above.

It is apparent from FIG. 5 that the second container 21 has been removed, which is to say unscrewed here, from the first container. A hypodermic needle 39, which is screwed into the coupling element 15, designed as a Luer connection here, by way of a known shoulder comprising an external thread, is connected to the first coupling element 15. Here, the stopper 11 is located in a position in which all air fractions have been expelled from the cavity 5, and preferably also from the hypodermic needle 39. Thus, a syringe is present here, which is prepared for an injection.

It was assumed in the figures, as mentioned above, that the second container 3 is a syringe that can be coupled to a hypodermic needle 39 via a coupling element 15.

However, it shall be pointed out here again that the first container 3 can also be designed as a carpule, which can be coupled to a second container 21 via a coupling device so as to dissolve a lyophilizate present in the carpule or in the second container of the carpule by way of a solvent, which is provided in the respective other container. The carpule can then be used in an injection system referred to as a pen, for example, or in an infusion device.

In light of the above, it is essential that the second container 21 has an elastic wall at least in regions. Ultimately, it can also take on any arbitrary shape, which is to say cylindrical, for example. It is thus not absolutely necessary to provide a container designed as a tube here.

It is furthermore important that the coupling device 31 must be designed such that a tight fluid connection is created between the two cavities in the containers. Since Luer systems are common, such a system should absolutely be preferred, which is to say a Luer connection as the first coupling element 15, and a second container comprising a shoulder provided with an external thread, which serves as the second coupling element 29.

The following shall be noted with respect to the function of the system for preparing an injection and for carrying out the method.

It is apparent from the explanations with respect to FIGS. 1 to 5 that, when carrying out the method for preparing an injection, the two containers 3 and 21 of a system 1, which comprise differing substances, and in particular a lyophilizate and a solvent, initially must be connected to one another such that a tight fluid connection is created between the cavities 5 and 23 enclosed by the containers 3 and 21. This is carried out by way of a coupling device 31, which comprises a first coupling element 15 on the first container 3 and a second coupling element 29 on the second container 21. After the containers have been coupled and a fluid connection been established, the liquid medium, this being the solvent, is supplied to the powdered medium, this being the lyophilizate. In the exemplary embodiment of the system described here, a solvent 25 from the second container 21 is introduced into the cavity 5 of the first container 3, in which the lyophilizate 17 is located. The transfer of the solvent 25 into the first container 3 is further promoted by the second container 21 having a variable volume, and preferably a wall 35, which is designed to be elastic at least in regions, and by the second container 21 being initially expanded by an air volume 19 from the first container 3.

In connection with FIGS. 3 and 4, it was described that initially the stopper 11 is moved upward in the direction of the arrow 33 inside the first container 3 so as to generate a positive pressure in the second container 21. As a result of the positive pressure and the displacement of the stopper 11 inside the first container 3 in the direction of the arrow 37 (see FIG. 4), the solvent 25 is transferred from the second container 21 into the cavity 5 of the first container 3.

However, it is also possible, proceeding from the situation according to FIG. 2, to displace the stopper 11 downward in the direction of the arrow 37, as is shown in FIG. 4, inside the first container 3, and to transfer the solvent 25 present in the second container 21 into the first container 3. In this case, the volume inside the second container 21 is decreased, as a comparison of FIGS. 2 and 4 shows.

The lyophilizate 17 is dissolved by moving and/or shaking the first container 3 containing the lyophilizate 17 and the solvent 25. The dissolution of the lyophilizate 17, as well as the thorough mixing of the substances, can be promoted by moving the stopper 11 up and down inside the first container 3 in direction of the longitudinal axis thereof. The stopper 11 can be moved up and down so far that the substances to be mixed flow through the coupling region between the two containers 3 and 21. Since, in general, a relatively small flow cross-section is present here, particularly good thorough mixing of the two substances takes place. In this way, a dissolution of a lyophilizate in a diluent is also promoted. During an upward movement of the stopper 11 in the direction of the arrow 33 (FIG. 3), the volume in the second container 21 increases, whereby the same is expanded, as shown in FIG. 3.

The movement of the stopper 11 is facilitated by the second container 21 having a variable volume and thus, at the most, meeting an upward movement of the stopper 11 with little counter-pressure.

It is known to couple two containers to one another to produce an injection solution, and to suction a liquid from one container into another container, for example, so as to mix the liquid there with a lyophilizate or with another liquid. In the system described here, a liquid flows through a coupling region between two containers, for example so as to make contact with another liquid or a lyophilizate. The resultant solution can be pushed back into the container, in which the suctioned liquid was initially located, through the coupling site so as to mix the two liquids or the dissolved lyophilizate. During the multiple up and down movements of a stopper 11 in direction of the longitudinal axis thereof inside the first container 3, the substances to be mixed pass through the coupling region multiple times so as to flow between the two containers 3 and 21. A liquid from the first container thus reaches the second container, which originally contained the liquid serving as the solvent or diluent.

Since it is preferably provided here that the amounts of the substances in the two containers for generating an injection solution are exactly matched to one another, the solution can be pushed out of the first and into the second container, which is easily possible due to the at least one elastic wall region thereof. This is a distinguishing feature compared to other systems, in which one container provides a large amount of liquid, which is suctioned into another container for multiple injections. In these known systems, it is important that the medium present in the container, which is suctioned into different syringes or carpules, is not contaminated under any circumstances, so that the injection solution to be generated is not contaminated even when the container is used multiple times.

The first container 3 is preferably a single-chamber syringe or carpule comprising the lyophilizate. Such syringes and carpules are known. The lyophilizate can be dissolved in a simple manner by way of a solvent by coupling the syringe or carpule to a second container 21 via a coupling device 31, wherein a fluid connection is created. The second container 21 has a variable volume, which is implemented, in particular, by an at least regionally elastic wall 35. The second container 21 is particularly preferably designed as a tube, which can be coupled to the first container, which is to say the syringe or carpule, in a simple manner. This tube can replace conventional containers, which usually contain a solvent and are coupled to the syringe or carpule. It is thus very easily possible to utilize the advantages of the second container 21 having a variable volume so as to dissolve a lyophilizate in conventional syringes and carpules, or so as to mix two different liquids with one another.

From the explanations, it becomes apparent that the at least one regionally elastic wall of a container, in particular of a tube, is an essential aspect of the invention. The wall is expanded to provide an injection solution, whereby the at least one regionally elastic wall is extended. The restoring forces of this wall cause a medium present in the second container to be expelled from the container under positive pressure and into the first container, whereby this medium mixes optimally with that in the first container. For example, a liquid jet from the second container impinges at a high speed and with high energy on a powdered substance in the first container, whereby this mixes optimally with the liquid. By nature, this phenomenon also arises when the liquid exiting the second container with high energy and at a high speed impinges on a further liquid in the first container.

Moreover, it is possible in a simple manner, by displacing the stopper 11 in the first container 3 in the direction of the second container and in the opposite direction, to allow the media present in the containers to pass through a fluid connection between the containers. During a back and forth movement of the stopper, a liquid flows back and forth through the fluid connection, together with a powder to be mixed or a further liquid to be mixed. Since this fluid connection has a smaller cross-section than the two containers, the substances pass through the fluid connection at a high speed, which brings about optimal mixing of the substances, and in particular good dissolution of a lyophilizate by way of a solvent or diluent.

Especially the at least regionally elastic wall is of great advantage since the wall is able to expand and retract during a back and forth movement of the stopper, and thus promotes the mixing of the substances. The wall poses relatively little resistance to an expansion of the second container.

In light of the above, it is shown that preferably a maximum of 100 ml is selected for the volumes of the first and second containers, and in particular of a syringe or cartridge and a tube. Volumes of 1 ml to 50 ml, and in particular of 1 ml to 10 ml or 15 ml, have proven particularly useful.

The latter volumes of the first container, or of the syringe or a carpule, and of the second container, or of the tube, yield particularly compact designs for the system for preparing an injection or a combination of syringe or a carpule on the one hand, and a tube on the other hand.

This embodiment is thus characterized by particularly good handling, which is also very manageable for patients.

1-18. (canceled)

19. A system for preparing an injection, the system comprising
a first container defining a first cavity for a first substance and having a rigid outer wall and two ends;
a movable stopper provided on one side of the first cavity which sealingly closes the first cavity and is displaceable inside the first container; and
a second container enclosing a second cavity for a second substance, the second container being a tube,
wherein the first container and the second container are connected to one another such that the first and second cavities are in fluid connection with one another,
wherein the second container includes an at least regionally elastic wall so as to have a variable volume, the at least regionally elastic wall operable to build a pressure in the second container.

20. The system according to claim 19, wherein one of the first and second substances is solid, and the other of the first and second substances is liquid.

21. The system according to claim 19, wherein the first and second substances are liquid.

22. The system according to claim 20, wherein the first substance is a lyophilizate, and the second substance is a solvent for the lyophilizate.

23. The system according to claim 22, wherein a lyophilizate is present in the first container, and the solvent for the lyophilizate is present in the second container.

24. The system according to claim 19, wherein the first container is a syringe or carpule.

25. The system according to claim 19, further comprising an air volume present in the first container.

26. The system according to claim 20, further comprising a coupling device implemented as a screw connection, the coupling device comprises a first coupling element attached to the first container and a second coupling element provided on the second container.

27. The system according to claim 26, wherein the first coupling element on the first container is a Luer connection, and the second coupling element comprises an external thread that can be screwed into the Luer connection.

28. The system according to claim 19, configured and designed such that an amount of the second substance is provided in the second container for an amount of a first substance in the first container, so as to provide an amount of an injection solution for an injection, wherein the volume of the first container is matched to the amount of the first substance and/or the volume of the second container is matched to the amount of the second substance.

29. The system according to claim 19, configured and designed such that a number of containers comprising differing amounts of a substance is provided so as to be combined with a number of containers comprising differing amounts of a second substance, wherein a first container including the first substance is brought in fluid connection with a second container including the second substance, in which the amount of the second substance is matched exactly to the amount of the first substance in the first container so as to provide an injection solution for an injection.

30. A combination of a syringe or carpule and a tube comprising at least one regionally elastic wall, the combination selected such that a volume provided in the syringe and a volume provided in the tube are matched to one another such that the syringe is able to receive a defined amount of a first substance and the tube is able to receive a defined amount of a second substance, and at least a partial volume from the syringe or carpule is introducible into the tube, so as to build a positive pressure in the tube and expand the at least one regionally elastic wall.

31. A method for preparing an injection, by way of a system according to claim 19, the method comprising:
coupling the first container and second container having the variable volume so as to implement a fluid connection, and
mixing the first and second substances in the first and second containers, respectively, by shaking and/or moving, including multiple times, the stopper provided in the first container,
wherein the variable volume in the second container is used to mix the substances.

32. The method according to claim 31, wherein a partial volume is displaced out of the first container by way of the stopper and into the second container, whereby the at least one regionally elastic wall of the second container is expanded against an elastic restoring force.

33. The method according to claim 31, wherein the second substance in the second container enters the first container at a high speed and/or with high energy when the stopper is pulled back in the container due to pressure that is built by the at least regionally elastic wall of the second container and the restoring force thereof.

34. The method according to claim 31, wherein one of the first and second substances is solid and the other is liquid.

35. The method according to claim 31, wherein both of the first and second substances are liquid.

36. The method according to claim 31, wherein the coupling takes place by screwing together a Luer system.
